(19) United States
(12) Patent Application Publication
(10) Pub. No.: US 2026/0115499 A1
(43) Pub. Date: Apr. 30, 2026

(54) NOISE-MINIMIZING STRUCTURE OF HIGH-INTENSITY FOCUSED ULTRASOUND GENERATION DEVICE

(71) Applicant: JEISYS MEDICAL INC., Seoul (KR)

(72) Inventors: Won Ju Yi, Seoul (KR); Eun Kyung Gong, Seoul (KR); Soo Min Hwang, Seoul (KR); Dong Hwan Kang, Seoul (KR)

(73) Assignee: JEISYS MEDICAL INC., Seoul (KR)

(21) Appl. No.: 19/004,495

(22) Filed: Dec. 30, 2024

Related U.S. Application Data
(63) Continuation of application No. PCT/KR2023/008042, filed on Jun. 12, 2023.

(30) Foreign Application Priority Data
Jul. 5, 2022 (KR) 10-2022-0082474
Jul. 7, 2022 (KR) 10-2022-0083888

(51) Int. Cl. A61N 7/02

(52) U.S. Cl. CPC A61N 7/02

(57) ABSTRACT

A high-intensity focused ultrasound generation device includes an integrated electrode including a ultrasound radiation frame, a transducer holder, and a transducer to allow electromagnetic interference (EMI) noise having focused only on the transducer to flow into the ultrasound radiation frame so as to minimize noise and thus is advantageous in that a signal loss due to the noise is prevented and an RF signal may be more stably applied. Furthermore, the ultrasound radiation frame, the transducer holder, and the transducer are included in the integrated electrode and thus a negative electrode of a power supplier may be connected to a negative electrode of a singular transducer or the ultrasound radiation frame so that a circuit structure may be very simple compared to a structure connected to each of a plurality of transducers.

### CROSS-REFERENCE TO RELATED APPLICATIONS

The present application is a continuation of International Patent Application No. PCT/KR2023/008042, filed on Jun. 12, 2023, which claims the benefit of priority to Korean Patent Application Nos. 10-2022-0082474 filed on Jul. 5, 2022 and 10-2022-0083888 filed on Jul. 7, 2022. The disclosures of the above-listed applications are hereby incorporated by reference herein in their entirety.

### FIELD

The present disclosure relates to a noise-minimization structure of a high-intensity focused ultrasound generation device in which a plurality of transducers are individually mounted on an ultrasonic radiation frame by a transducer holder, and the transducers, the transducer holder, and the ultrasonic radiation frame are grounded and function as an integral electrode, so that noise focused only on the transducers can be minimized.

### BACKGROUND

In general, a high-intensity focused ultrasound (HIFU) generator is a device that focuses ultrasound generated from a transducer to generate high-intensity ultrasound energy, irradiates it to the patient's affected area, and increases the temperature of the affected area, thereby treating the affected area without surgical intervention.

In a conventional high-intensity focused ultrasound generation device, when tens or hundreds of transducers are used, a plurality of transducers is mounted on a front of an ultrasound radiation frame, and then the entire front of the ultrasound radiation frame is coated with glue to form a waterproof layer, thereby fixing the plurality of transducers by the waterproof layer and preventing water leakage.

However, since the ultrasonic energy generated forward from the transducers is absorbed by the waterproof layer, there is a problem that the input voltage needs to be increased to compensate for this, and there is also a problem that EMI (Electro Magnetic Interference) noise is generated from a plurality of transducers and that the ultrasonic radiation frame needs to be replaced even in the case that one of the plurality of transducers breaks down.

### SUMMARY

In an exemplary embodiment, the present disclosure provides a high-intensity focused ultrasound generation device. The device includes: an ultrasonic radiation frame having a coupling hole formed therein; a transducer holder inserted into the coupling hole and detachably coupled through the ultrasonic radiation frame; and a transducer mounted on the transducer holder. The ultrasonic radiation frame, the transducer holder, and the transducer are electrically connected to each other to form an integrated electrode.

### BRIEF DESCRIPTION OF THE FIGURES

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a perspective view illustrating a head module of a high-intensity focused ultrasound generation device according to a first embodiment of the present disclosure.

FIG. 2 is an exploded perspective view illustrating a combined structure of an ultrasonic radiation frame and a transducer holder according to the first embodiment of the present disclosure.

FIG. 3 is a cross-sectional view illustrating a coupling structure of an ultrasonic radiation frame and a transducer holder according to the first embodiment of the present disclosure.

FIG. 4 is an enlarged view of part A of FIG. 3.

FIG. 5 is a front perspective view of a transducer holder according to the first embodiment of the present disclosure.

FIG. 6 is a rear perspective view of the transducer holder shown in FIG. 5.

FIG. 7 is a diagram illustrating an electrode structure using a transducer holder according to a second embodiment of the present disclosure.

FIG. 8 is a rear view illustrating an ultrasonic radiation frame and an RF board according to a third embodiment of the present disclosure.

FIG. 9 is a cross-sectional view taken along line A-A of FIG. 8.

### DETAILED DESCRIPTION

Exemplary embodiments of the present disclosure provide a noise-minimizing structure for a high-intensity focused ultrasound generation device, which facilitates replacement and repair of a transducer and minimizes noise generation to improve stability.

A noise-minimizing structure of high-intensity focused ultrasound generation device according to an aspect of the present disclosure includes an ultrasonic radiation frame having a concave front surface and a plurality of coupling holes formed therein; a plurality of transducer holders respectively inserted into the plurality of coupling holes in a front of the ultrasonic radiation frame and detachably coupled through the ultrasonic radiation frame; and a plurality of transducers respectively mounted on the plurality of transducer holders with front surfaces thereof exposed, wherein the ultrasonic radiation frame is made of an insulating material, wherein the ultrasonic radiation frame, the transducer holder, and the transducer are each formed to have electrical conductivity and are electrically connected to each other as an integrated electrode.

At least one of a negative pole of the ultrasonic radiation frame or the transducer is connected to a negative pole of a power supply unit and grounded, and the transducers are respectively connected to a positive pole of the power supply unit.

The ultrasonic radiation frame, the transducer holder, and the transducer are formed of a conductive material.

At least some of the ultrasonic radiation frame, the transducer holder, or the transducer are formed of a non-conductive material, but have a surface coated with a conductive material.

The conductive material includes at least one of chromium, nickel, cadmium, iron, copper, platinum, gold, silver, lead, or an alloy thereof.

The transducer holder includes a head portion mounted on the front surface of the ultrasonic radiation frame and has a mounting groove formed into which the transducer is inserted and mounted; and a body portion that extends rearward from the head portion and penetrates the coupling hole and is coupled by a fastening member at a rear of the ultrasonic radiation frame.

The body portion of the transducer holder is formed with the current supply hole through which an electrode wire connected to the transducers passes through and is withdrawn to the rear of the ultrasonic radiation frame, and wherein a space between the current supply portion and the current supply hole is sealed with waterproof glue.

The head portion of the transducer holder is formed so that at least a part of a side surface of the mounting groove is opened.

The head portion of the transducer holder is formed with at least one support protrusion that protrudes from a bottom surface of the mounting groove and supports a lower surface of the transducer and form a space between the transducer and the bottom surface.

The head portion of the transducer holder is formed with a catch protrusion that protrudes from a bottom surface of the mounting groove and has a tip bent inward to prevent the transducer inserted into the mounting groove from being detached.

The body portion of the transducer holder includes a shaft portion that extends rearward from the head portion and is press-fitted into the coupling hole; and a screw portion that extends rearward from the shaft portion and penetrates the coupling hole and is coupled with the fastening member at the rear of the ultrasonic radiation frame.

The device further includes an RF board provided on the ultrasonic radiation frame, and in which the plurality of transducers are electrically connected to each other and supply RF power to the transducers.

The device further includes a plurality of electrode wires respectively connected to the plurality of transducers; and a plurality of board connectors respectively provided corresponding to the electrode wires in the RF board, and to which the electrode wires are detachably connected.

The plurality of board connectors are detachably connected to the RF board.

The device further includes a monitoring sensor provided on the RF board and configured to independently monitor operating states of the transducers by detecting the power supply states of the electrode wires respectively connected to the transducers.

The device further includes an insulating cover formed to cover an outer side of the RF board.

The device further includes a power supply device for supplying the RF power to the RF board; and a power cable for connecting the RF board and the power supply device and detachably coupled with the RF board.

The device further includes a probe coupled to a center of the ultrasonic radiation frame, wherein the RF board is provided on a remaining part of the rear of the ultrasonic radiation frame except for a coupling portion where the probe is coupled.

A noise-minimizing structure of high-intensity focused ultrasound generation device according to another aspect of the present disclosure includes an ultrasonic radiation frame having a probe arranged in a front center and a plurality of coupling holes formed around the probe; a plurality of transducer holders respectively inserted into the plurality of coupling holes in front of the ultrasonic radiation frame, penetrating the ultrasonic radiation frame, and detachably coupled at a rear of the ultrasonic radiation frame; and a plurality of transducers respectively mounted on open front surfaces of at least some of the plurality of transducer holders, wherein the transducer holder includes: a head portion mounted on a front surface of the ultrasonic radiation frame and has a mounting groove formed into which the transducer is inserted and mounted; and a body portion that extends rearward from the head portion and penetrates the coupling hole and is coupled by a fastening member at the rear of the ultrasonic radiation frame, wherein the head portion has a plurality of support protrusions that protrudes from a bottom surface of the mounting groove and supports a lower surface of the transducer and form a space between the transducer and the bottom surface, wherein the ultrasonic radiation frame, the transducer holder, and the transducer are each formed to have electrical conductivity and are electrically connected to each other as an integrated electrode, wherein at least one of a negative pole of the ultrasonic radiation frame or the transducer is connected to a negative pole of a power supply unit and grounded, wherein the transducers are respectively connected to a positive pole of the power supply unit, and wherein the ultrasonic radiation frame, the transducer holder, and the transducer are formed of a conductive material.

A noise-minimizing structure of high-intensity focused ultrasound generation device according to still another aspect of the present disclosure includes an ultrasonic radiation frame having a probe arranged in a front center and a plurality of coupling holes formed around the probe; a plurality of transducer holders respectively inserted into the plurality of coupling holes in front of the ultrasonic radiation frame, penetrating the ultrasonic radiation frame, and detachably coupled at a rear of the ultrasonic radiation frame; and a plurality of transducers respectively mounted on open front surfaces of at least some of the plurality of transducer holders, wherein the transducer holder includes: a head portion mounted on a front surface of the ultrasonic radiation frame and has a mounting groove formed into which the transducer is inserted and mounted; and a body portion that extends rearward from the head portion and penetrates the coupling hole and is coupled by a fastening member at the rear of the ultrasonic radiation frame, wherein the head portion has a plurality of support protrusions that protrudes from a bottom surface of the mounting groove and supports a lower surface of the transducer and form a space between the transducer and the bottom surface, wherein the ultrasonic radiation frame, the transducer holder, and the transducer are each formed to have electrical conductivity and are electrically connected to each other as an integrated electrode, wherein at least one of a negative pole of the ultrasonic radiation frame or the transducer is connected to a negative pole of a power supply unit and grounded, wherein the transducers are respectively connected to a positive pole of the power supply unit, and wherein at least some of the ultrasonic radiation frame, the transducer holder, or the transducer are formed of a non-conductive material, but have a surface coated with a conductive material.

A noise-minimizing structure of high-intensity focused ultrasound generation device according to still another aspect of the present disclosure includes an ultrasonic radiation frame having a probe arranged in a front center and a plurality of coupling holes formed around the probe; a plurality of transducer holders respectively inserted into the plurality of coupling holes in front of the ultrasonic radiation frame, penetrating the ultrasonic radiation frame, and detachably coupled at a rear of the ultrasonic radiation frame; and a plurality of transducers respectively mounted on open front surfaces of at least some of the plurality of transducer holders, wherein the ultrasonic radiation frame, the transducer holder, and the transducer are each formed to have electrical conductivity and are electrically connected to each other as an integrated electrode, further comprising: an RF board provided on the ultrasonic radiation frame and supply RF power to the transducers; a plurality of electrode wires respectively connected to the plurality of transducers; a plurality of board connectors respectively provided corresponding to the electrode wires in the RF board, and to which the electrode wires are detachably connected; and a monitoring sensor provided on the RF board and configured to independently monitor operating states of the transducers by detecting the power supply states of the electrode wires respectively connected to the transducers.

A noise-minimizing structure of high-intensity focused ultrasound generation device according to still another aspect of the present disclosure includes an ultrasonic radiation frame having a concave front surface and a plurality of coupling holes formed therein; a plurality of transducer holders respectively inserted into the plurality of coupling holes in a front of the ultrasonic radiation frame and detachably coupled through the ultrasonic radiation frame; and a plurality of transducers respectively mounted on the plurality of transducer holders with front surfaces thereof exposed, wherein the ultrasonic radiation frame is made of an insulating material, wherein the ultrasonic radiation frame, the transducer holder, and the transducer are each formed to have electrical conductivity and are electrically connected to each other as an integrated electrode, further comprising: an RF board provided on the ultrasonic radiation frame, and in which the plurality of transducers are electrically connected to each other and supply RF power to the transducers; a plurality of electrode wires respectively connected to the plurality of transducers; a plurality of board connectors respectively provided corresponding to the electrode wires in the RF board, and to which the electrode wires are detachably connected; a monitoring sensor provided on the RF board and configured to monitor operating states of the transducers; and an insulating cover formed to cover an outer side of the RF board, wherein the transducer holder includes: a head portion mounted on a front surface of the ultrasonic radiation frame and has a mounting groove formed into which the transducer is inserted and mounted; and a body portion that extends rearward from the head portion and penetrates the coupling hole and is coupled by a fastening member at the rear of the ultrasonic radiation frame, wherein the body portion of the transducer holder is formed with the current supply hole so that the current supply portion passes through and is withdrawn to the rear of the ultrasonic radiation frame, and wherein the body portion of the transducer holder is formed with the current supply hole through which an electrode wire passes through and is withdrawn to the rear of the ultrasonic radiation frame.

Hereinafter, exemplary embodiments of the present disclosure will be described with reference to the attached drawings.

A high-intensity focused ultrasound generation device according to an embodiment of the present disclosure is a device using high-intensity focused ultrasound (HIFU). The high-intensity focused ultrasound generation device includes a transducer array in which tens or hundreds of transducers are radially arranged, and may not only treat the affected area of a patient with a tumor, but also stimulate the brain to treat Alzheimer's disease or depression, and may also increase immunity by applying heat to a specific area.

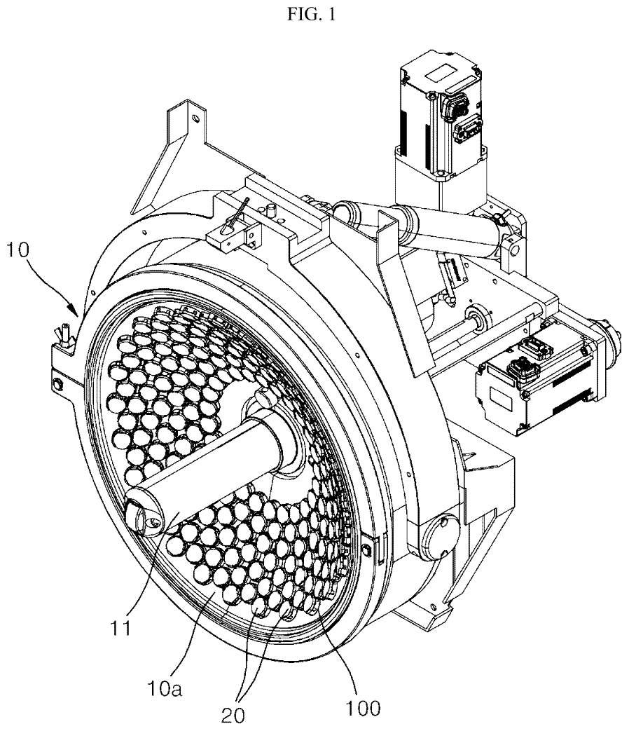

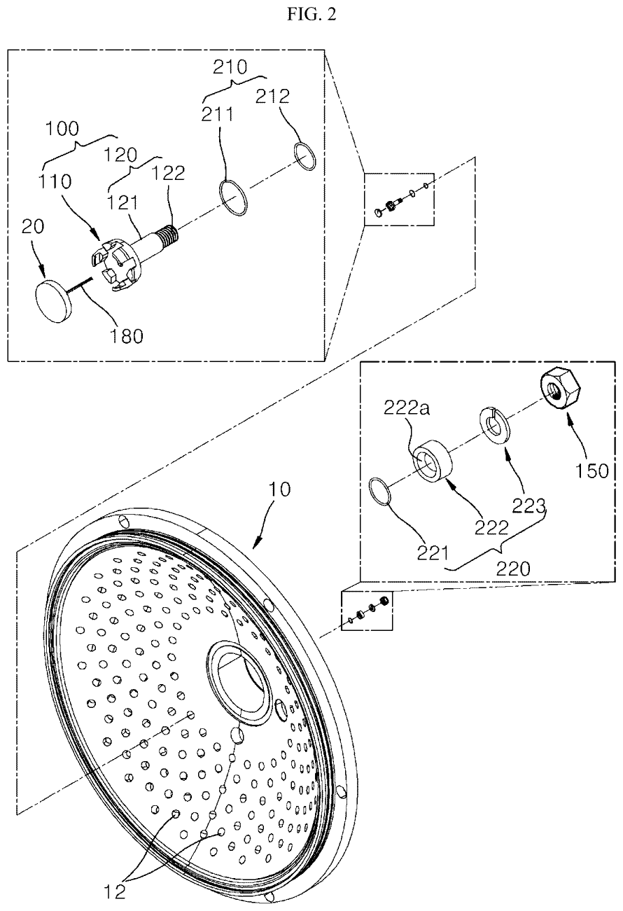

FIG. 1 is a perspective view illustrating a head module of a high-intensity focused ultrasound generation device according to a first embodiment of the present disclosure. FIG. 2 is an exploded perspective view illustrating a combined structure of an ultrasonic radiation frame and a transducer holder according to the first embodiment of the present disclosure.

Referring to FIGS. 1 and 2, a head module of a high-intensity focused ultrasound generation device includes an ultrasonic radiation frame 10, a plurality of transducers 20, and a plurality of transducer holders 100.

The ultrasonic radiation frame 10 is formed to have electrical conductivity. The ultrasonic radiation frame 10 is formed of a conductive material, for example. The conductive material may be a metal having electrical conductivity, for example. However, the present disclosure is not limited thereto, and the ultrasonic radiation frame 10 may be formed of a non-conductive material and then the surface may be coated with the conductive material. In the case that the ultrasonic radiation frame 10 is coated with the conductive material, the thickness of the coating layer is set to a skin depth that may conduct electricity.

The ultrasonic radiation frame 10 has a probe 11 attached to a center of a front 10a, and a plurality of coupling holes 12 are arranged radially around the probe 11. The ultrasonic radiation frame 10 is formed in a plate shape with a concave front so as to focus ultrasound radiated from the plurality of transducers 20 and radiate them to one position.

The plurality of coupling holes 12 are through holes formed at a predetermined interval from each other. The number of the coupling holes 12 is set according to the number of the transducers 20.

The plurality of transducers 20 is formed to have electrical conductivity. The plurality of transducers 20 includes a piezoelectric element. The transducer 20 generates ultrasound when voltage is applied. The transducer 20 is formed in a disc shape as an example. The transducers 20 are arranged in radial patterns in dozens or hundreds to form a transducer array. The number of the transducers 20 may be set according to the ultrasonic energy to be radiated.

The transducers 20 are coated with an electrode material having a conductive material on each surface. That is, the inside of each of the transducers 20 is made of a piezoelectric element material, and the surface is coated with an electrode material.

The transducer holders 100 are formed to have electrical conductivity. In this embodiment, the transducer holders 100 may be formed of a non-conductive material and may have their respective surfaces coated with a conductive material. That is, the transducer holder 100 is formed of a resin material and having its surface coated with the conductive material as an example.

The conductive material includes at least one of chromium, nickel, cadmium, iron, copper, platinum, gold, silver, lead, or an alloy, for example. However, the present disclosure is not limited thereto, and any material having electrical conductivity may be applied.

In this embodiment, the surface of the transducer holder 100 may have a chromium coating layer 170 formed as an example.

The transducer holder 100 is detachably coupled to each of the plurality of coupling holes 12 of the ultrasonic radiation frame 10.

The transducer 20 is coupled to each of the transducer holders 100. The transducer holder 100 and the transducer 20 are integral electrodes that are electrically connected by contacting each other. The electrodes are connected to a power supply unit described below and are supplied with power.

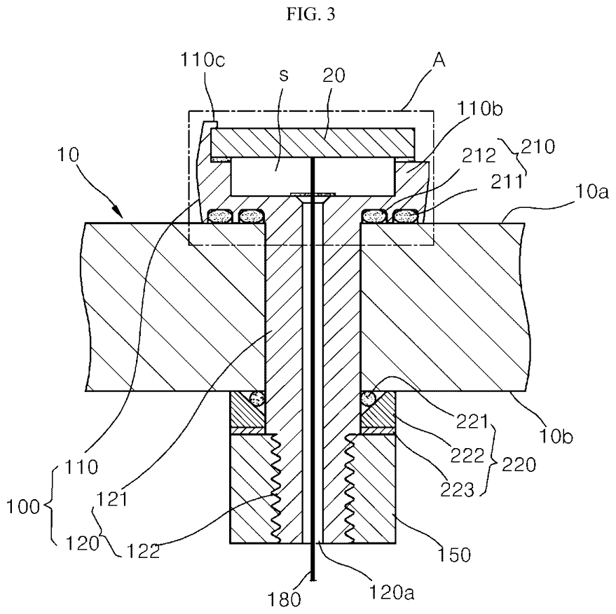

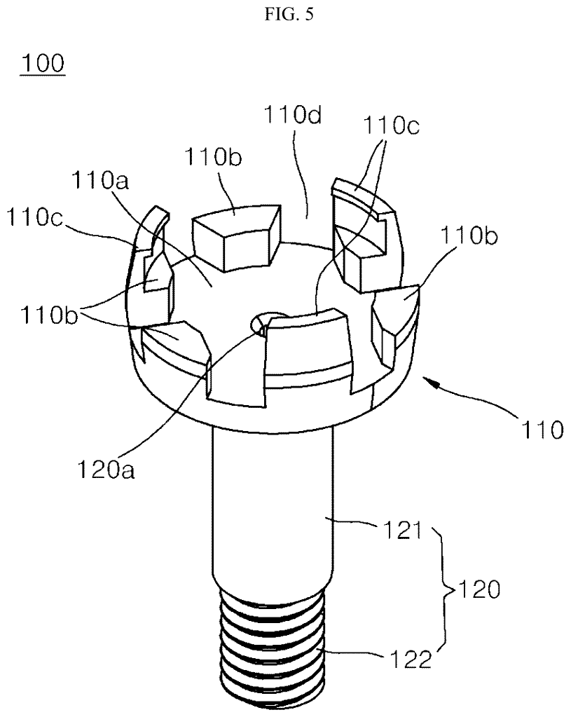

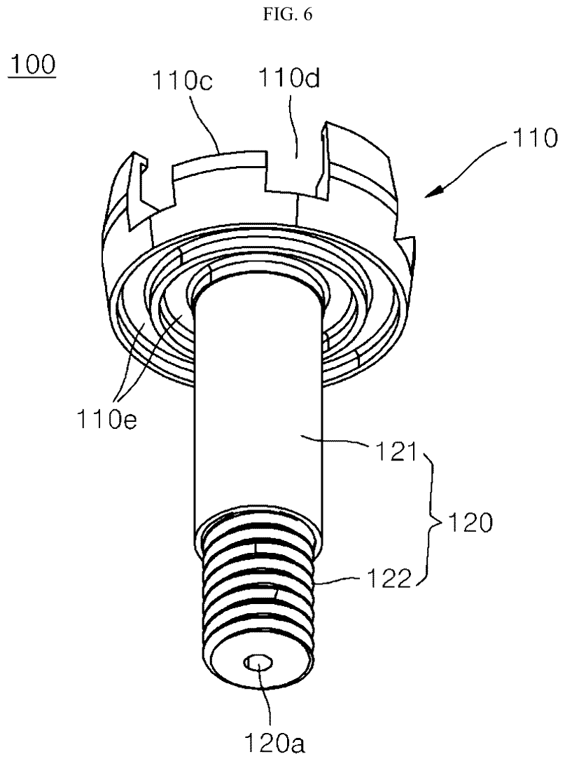

Referring to FIGS. 3 to 6, the transducer holder 100 includes a head portion 110 having a mounting groove 110a formed in which the transducer 20 is inserted and mounted, and a body portion 120 that extends to a rear of the head portion 110 and is coupled to the coupling holes 12.

The head portion 110 is formed to have a diameter greater than the coupling hole 12 so that the head portion 110 is mounted on the front 10a of the ultrasonic radiation frame 10. The mounting groove 110a, a support protrusion 110b, a catch protrusion 110c, and an opening 110d are formed in the head portion 110.

The mounting groove 110a is formed with an open front in front of the head portion 110 so that the transducer 20 is mounted therein.

The support protrusion 110b is formed to protrude forward from the bottom surface of the mounting groove 110a to a predetermined height so as to support the lower surface of the transducer 20. The support protrusion 110b forms a space S between the lower surface of the transducer 20 and the bottom surface of the mounting groove 110a, thereby forming a passage through which the electrode wire 180 coupled to the transducer 20 passes, thereby stably implementing the electrode structure, and also enables vibration of the transducer 20 so as to maximize the vibration wave energy of the transducer 20. The above support protrusion 110b is composed of multiple pieces and is formed to be spaced apart from each other by a predetermined interval, as an example. However, the present disclosure is not limited thereto, and the support protrusion 110b may also be provided with one piece in the center of the bottom surface of the mounting groove 110a. In addition, the support protrusion 110b may also be formed integrally with the head portion 110.

The catch protrusion 110c is formed to protrude from the bottom surface of the mounting groove 110a and have a tip bent inward, thereby preventing the transducer 20 inserted into the mounting groove 110a from being detached. The tip of the catch protrusion 110c may be changed to a shape that may prevent the transducer 20 from being detached, such as a hook shape. The catch protrusion 110c is formed in a plurality of pieces spaced apart from each other by a predetermined interval. In this embodiment, some of the plurality of catch protrusions 110c may be formed by protruding from the support protrusion 110b.

The opening 110d is a part formed by being cut open in the side of the mounting groove 110a. The opening 110d has the advantage of easy assembly. In addition, the opening 110d allows the transducer 20 to vibrate inside the mounting groove 110a, thereby maximizing the vibration wave energy of the transducer 20.

It is preferable that the body portion 120 is formed to extend rearwardly from the head portion 110 and penetrates the coupling hole 12. The body portion 120 is formed to have a diameter smaller than that of the head portion 110. A current supply hole is formed in the center of the body portion 120 so that a current supply portion, described below, may pass through the current supply hole.

The body portion 120 includes a shaft portion 121 and a screw portion 122.

The shaft portion 121 is formed in a cylindrical shape so as to extend rearwardly from the head portion 110 and be pressed into the coupling hole 12.

The screw portion 122 is formed with threads on the outer surface so as to extend rearwardly from the shaft portion 121 and be fastened by a fastening member 150.

The fastening member 150 is preferably a nut, but is not limited thereto.

Meanwhile, FIG. 3 is a cross-sectional view illustrating a coupling structure of an ultrasonic radiation frame and a transducer holder according to the first embodiment of the present disclosure.

The adhesive material may be a flexible glue. A flexible glue layer 200 is formed between at least one of the rear surface or the side surface of the transducer 20 and the head portion 110 by the flexible glue. The flexible glue may use a silicone or epoxy-based glue, and any flexible material may be used.

In this embodiment, the flexible glue layer 200 may be formed between the rear surface of the transducer 20 and the support protrusion 110b. However, the present disclosure is not limited thereto, and the flexible glue layer 200 may also be formed between the side surface of the transducer 20 and the inner surface of the catch protrusion 110c. That is, the flexible glue layer 200 may be applied to any position so long as the flexible glue layer 200 does not cover the front of the transducer 20.

Since the transducer 20 is fixed by being adhered to the transducer holder 100 by the flexible glue, the position of the transducer 20 inside the transducer holder 100 is fixed while the transducer 20 may vibrate, so that the vibration wave energy loss of the transducer 20 may be minimized. In addition, since the glue is not applied to the front of the transducer 20, the loss of ultrasonic energy radiated forward from the transducer 20 may be prevented. That is, since the flexible glue layer 200 is formed only on the rear surface or the side surface of the transducer 20, it does not cover the front of the transducer 20, and thus there is no restriction on the radiation of ultrasonic energy through the front.

In addition, the space between the transducer holder 100 and the ultrasonic radiation frame 10 is sealed by a sealing member.

The sealing member includes a first sealing member 210 that seals between the head portion 110 of the transducer holder 100 and the front 10a of the ultrasonic radiation frame 10, and a second sealing member 220 that seals between the body portion 120 and the rear 10b of the ultrasonic radiation frame 10.

The first sealing member 210 may include two first and second O-rings 211 and 212 inserted and combined into the rear of the head portion 110. However, the present disclosure is not limited thereto, and the number of the first sealing members 210 may be changed and applied in various ways. In addition, the first sealing member 210 may be applied to any structure that is made of various materials such as silicone and rubber other than an O-ring and may seal.

It is preferable that the first O-ring 211 and the second O-ring 212 are formed with different diameters. The first O-ring 211 and the second O-ring 212 are inserted into a ring-shaped groove 110e formed at the rear of the head portion 110 and are sealed by being pressed against the front surface 10a of the ultrasonic radiation frame 10.

The second sealing member 220 includes a third O-ring 221 that is inserted into the shaft portion 121 of the body portion 120, and an O-ring pressurizing member 222 that is inserted into the shaft portion 121 from the rear of the third O-ring 221 and presses the third O-ring 221 against the rear surface 10b of the ultrasonic radiation frame 10.

The O-ring pressurizing member 222 is formed in a ring shape, and an inclined surface 222a is formed on the front surface so that a part of the third O-ring 221 is settled.

The second sealing member 220 may further include a washer 223 provided between the O-ring pressurizing member 222 and the fastening member 150. The washer 223 is not an essential component of the second sealing member 220 and may be additionally included. The washer 223 may seal the third O-ring 221 and the O-ring pressurizing member 222 and may serve to hold the transducer holder 100.

The second sealing member 220 may be applied to any structure that is made of various materials such as silicone or rubber other than an O-ring or washer and may be sealed.

In addition, a waterproof glue layer 250 is formed between the electrode wire hole 120a of the transducer holder 100 and the electrode wire 180 described below. The waterproof glue may be the same as the flexible glue. In addition, the waterproof glue layer 250 may be formed to fill the entire separation space S with the waterproof glue.

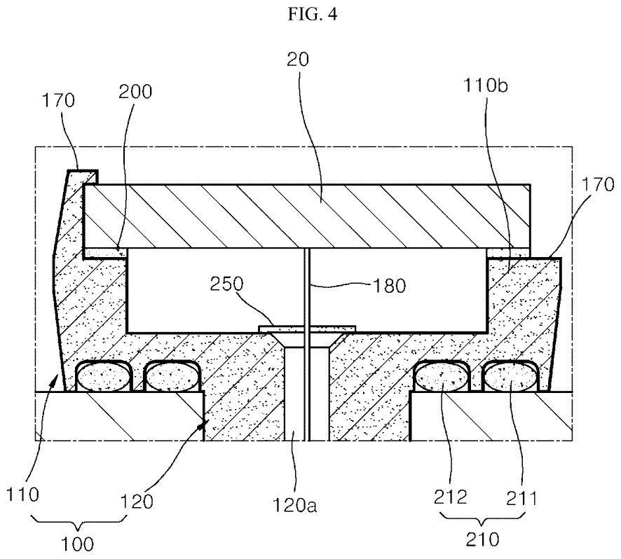

Meanwhile, referring to FIG. 4, the electrode structure using the transducer holder 100 is described as follows.

In the present disclosure, all of the ultrasonic radiation frame 10, the transducer holder 100, and the transducer 20 are formed to have electrical conductivity, and are an integral electrode that is electrically connected by contacting each other.

The ultrasonic radiation frame 10 is connected to a negative pole of the power supply unit for supplying power and is grounded. However, the present disclosure is not limited thereto, and positive and negative poles of the power supply unit may of course be connected to the transducers 20, respectively.

The transducers 20 are respectively connected to the positive pole of the power supply unit. The transducers 20 are connected to the power supply unit through an electrode line 180. The electrode line 180 may be connected to either the transducer holder 100 or the transducer 20. In the present embodiment, the electrode line 180 may be a wire that is soldered to the center of the rear surface of the transducer 20. The above electrode wire 180 is extended to the rear of the ultrasonic radiation frame 10 through the electrode wire hole of the transducer holder 100 and connected to a separate circuit board. However, the present disclosure is not limited thereto, and any power supply unit that may supply power, such as a pin or connector, may be applied.

The ultrasonic radiation frame 10 is grounded, and the transducers 20 are configured to receive power from the electrode wire 180, so that current is supplied to the transducers 20 by a potential difference applied to each of the ultrasonic radiation frame 10 and the transducers 20.

As described above, in the present disclosure, the ultrasonic radiation frame 10, the transducer holder 100, and the transducer 20 form an integrated electrode, and the ultrasonic radiation frame 10 is configured to be grounded, so that when both the positive and negative poles are connected to the transducers 20, noise that was concentrated only on the transducers 20 may flow to the ultrasonic radiation frame 10, thereby minimizing noise.

In addition, when noise generation is minimized, signal loss due to noise is prevented, so that an RF signal may be applied more stably, so that high-intensity focused ultrasound may be irradiated to the exact lesion position of the patient.

Furthermore, in the case that a plurality of transducers 20 are individually grounded, there is a problem that the structure becomes complicated because the number of electrode lines for grounding increases, but since the negative pole of the power supply unit may be connected to only one ultrasonic radiation frame 10 and grounded, the power connection and circuit structure may be simplified.

In addition, by using the transducer holder 100 and the transducer 20 as an integrated electrode, the sound wave loss may be minimized when the transducer vibrates.

Therefore, since there is no need to solder the electrode wire on the front side of the transducer 20, leakage due to the soldering structure on the front side of the transducer 20 may be prevented. That is, leakage into the interior of the front side of the transducer 20 that is exposed on the front side of the ultrasonic radiation frame 10 and contacts the liquid may be prevented.

In addition, since there is no need to solder the electrode wire on the front side of the transducer 20, there is an advantage in that the electrode structure is simplified and damage to the transducer 20 may be prevented.

Furthermore, the high-intensity focused ultrasound generation device configured as described above may prevent water from leaking into the interior from the front surface of the ultrasonic radiation frame 10 by mounting a plurality of transducers 20 on the ultrasonic radiation frame 10 using the transducer holder 100, and sealing the transducers 20 and the transducer holder 100 by bonding them with the flexible glue, even in the case that glue is not applied to the entire front surface of the ultrasonic radiation frame 10.

In addition, since glue is not applied to the entire front surface of the ultrasonic radiation frame 10, the entire front surface of the transducers 20 is exposed, and loss of ultrasonic energy radiated forward from the transducer 20 may be prevented. In the conventional art, in the case that the front of the transducers 20 is covered by a glue layer, there is a problem that ultrasonic energy is absorbed by the glue layer, but in the present disclosure, since the entire front of the transducers 20 is exposed, this may be prevented.

Furthermore, since the transducer 20 is bonded with the flexible glue inside the transducer holder 100, the position of the transducer 20 is fixed and play is prevented, while the vibration of the transducer 20 is possible, so that the vibration wave energy loss of the transducer 20 may be reduced.

In addition, since the plurality of transducers 20 are individually mounted through the transducer holder 100, and the transducer holder 100 is detachably coupled to the ultrasonic radiation frame 10, there is an advantage in that the transducers 20 may be individually repaired and replaced.

Furthermore, since the plurality of transducers 20 are individually mounted through the transducer holder 100, there is an advantage in that the capacities of at least some of the plurality of transducers 20 may be configured differently. For example, it is possible to increase the capacities of the transducers arranged on the central side of the ultrasonic radiation frame 10, and of course, it is also possible to control the voltages applied to the plurality of transducers 20 differently.

In addition, since the space between the transducer holder 100 and the ultrasonic radiation frame 10 is sealed by a sealing member such as an O-ring, not only may water leakage from the front to the rear of the ultrasonic radiation frame 10 be prevented, but there is also an advantage in that the transducer holder may be easily attached and detached from the ultrasonic radiation frame.

Meanwhile, in the above embodiment, all the transducers 20 may be coupled to the coupling holes 12 of the ultrasonic radiation frame 10, but the present disclosure is not limited thereto, and it is of course also possible to provide the transducers 20 in only at least some of the coupling holes 12 depending on the capacity of the high-intensity focused ultrasonic generation device. In the case that the transducers 20 are provided in only at least some of the coupling holes 12, the transducer holders 100 are coupled to all of the coupling holes 12, and a holder cover for shielding the open front may be detachably coupled to some of the transducer holders 100 to which the transducers 20 are not coupled. The holder cover may be formed of a different material from the transducers 20 but with the same shape and may be coupled by glue. Accordingly, the number of transducers 20 mounted may be adjusted, thereby controlling the energy capacity of the high-intensity focused ultrasound generation device.

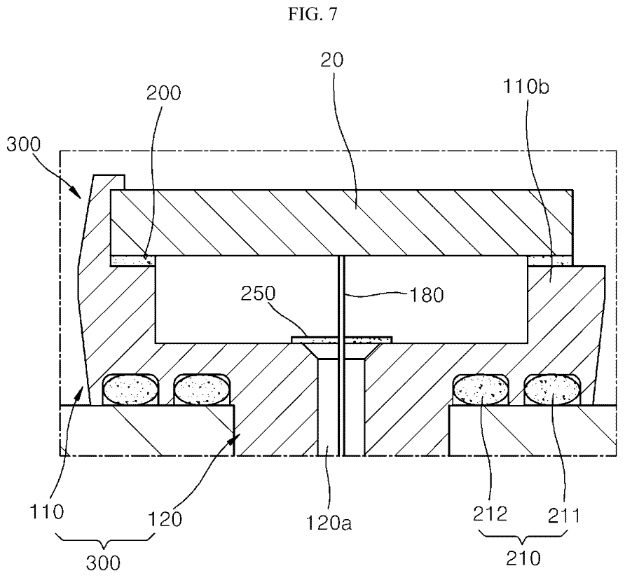

Meanwhile, FIG. 7 is a diagram illustrating an electrode structure using a transducer holder according to a second embodiment of the present disclosure.

Referring to FIG. 7, the electrode structure using the transducer holder according to the second embodiment of the present disclosure is different from the first embodiment in that the entire transducer holder 300 is an electrode formed of a conductive material, and the remaining configuration and operation are the same as the first embodiment, so a detailed description of the similar configuration will be omitted and the differences will be described based on the main points.

The transducer holder 300 is formed of the conductive material, and the structure or shape is applied to the above embodiment.

Any material having electrical conductivity, such as a metal, may be applied as the conductive material.

The surface of the transducer 20 is coated with an electrode material. Any material that may be used as an electrode, such as a metal such as silver, may be applied as the electrode material.

All of the ultrasonic radiation frame 10, the transducer holder 100, and the transducer 20 are formed to have electrical conductivity, and are an integral electrode that is electrically connected by contacting each other.

The ultrasonic radiation frame 10 is connected to the negative pole of the power supply unit for supplying power and is grounded.

The transducers 20 are respectively connected to the positive pole of the power supply unit. The transducers 20 are connected to the power supply unit through the electrode wire 180. The electrode wire 180 may be connected to either the transducer holder 100 or the transducer 20. In this embodiment, the electrode wire 180 may be a wire that is soldered to the center of the rear surface of the transducer 20. The electrode wire 180 is extended to the rear surface of the ultrasonic radiation frame 10 through the electrode wire hole of the transducer holder 100 and connected to a separate circuit board. However, the present disclosure is not limited thereto, and any power supply unit that may supply power, such as a pin or a connector, may be applied.

The transducer 20 is mounted on the support protrusion 110b, and the space S is formed between the transducer 20 and the bottom surface of the mounting groove of the transducer holder 100, so that the electrode wire 180 is prevented from contacting the surface of the transducer holder 300, and therefore, a short circuit does not occur.

The ultrasonic radiation frame 10 is grounded, and the transducers 20 are configured to receive power from the electrode wire 180, so that current is supplied to the transducer 20 by the potential difference applied to the ultrasonic radiation frame 10 and the transducers 20.

As described above, in the present disclosure, the ultrasonic radiation frame 10, the transducer holder 100, and the transducer 20 form an integral electrode, and the ultrasonic radiation frame 10 is configured to be grounded, in the case that both the positive and negative poles are connected to the transducers 20, noise that is concentrated only on the transducers 20 may flow to the ultrasonic radiation frame 10, thereby minimizing noise.

In addition, in the case that noise generation is minimized, signal loss due to noise is prevented, so that an RF signal may be applied more stably, so that high-intensity focused ultrasound may be irradiated to the exact lesion position of the patient.

Furthermore, in the case that a plurality of transducers 20 is individually grounded, there is a problem that the structure becomes complicated because the number of electrode lines for grounding increases, but since the negative pole of the power supply unit may be connected to only one ultrasonic radiation frame 10 and grounded, the power connection and circuit structure may be simplified.

In addition, since there is no need to solder the electrode wire on the front side of the transducer 20, leakage due to the soldering structure on the front side of the transducer 20 may be prevented. That is, leakage into the interior of the front side of the transducer 20 that is exposed on the front side of the ultrasonic radiation frame 10 and contacts the liquid may be prevented.

Furthermore, since there is no need to solder the electrode wire on the front side of the transducer 20, there is an advantage in that the electrode structure may be simplified and damage to the transducer 20 may be prevented. In addition, at least a portion of the side surface or the rear surface of the transducer 20 may be coated with a waterproof material to prevent corrosion or fluctuation of the output value due to water intrusion.

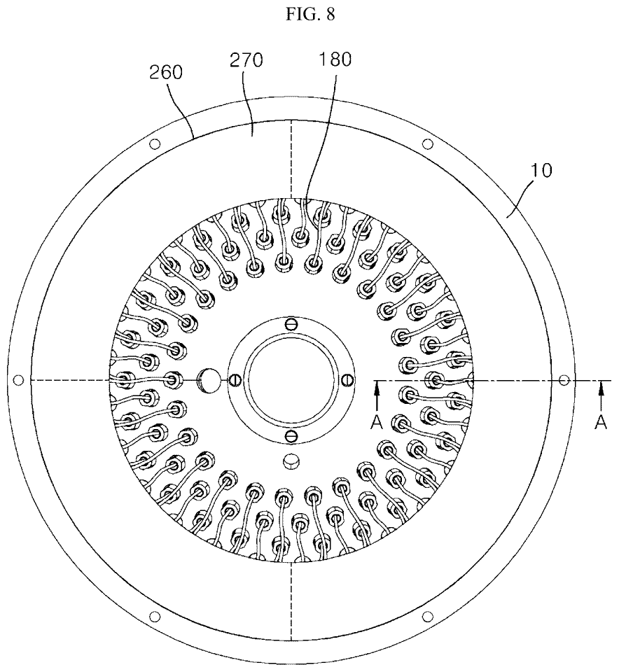

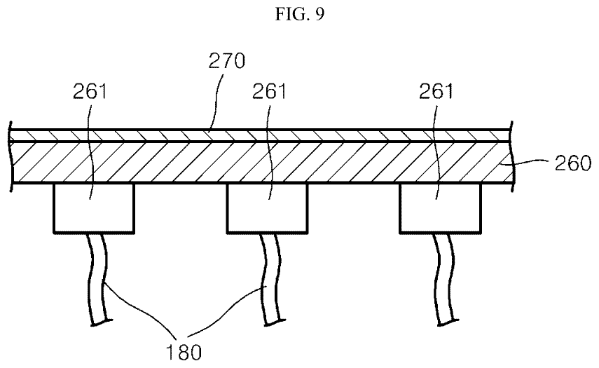

Meanwhile, FIG. 8 is a rear view illustrating an ultrasonic radiation frame and a RF board according to a third embodiment of the present disclosure. FIG. 9 is a cross-sectional view taken along line A-A of FIG. 8.

Referring to FIGS. 8 and 9, a high-intensity focused ultrasound generation device according to the third embodiment of the present disclosure is different from the first and second embodiments in that it further includes an RF board 260 that is provided on the ultrasonic radiation frame 10 and electrically connects the plurality of transducers 20 to each other and supplies RF power to the transducers 20, and the remaining configuration and operation are similar, so the following description will focus on the different configuration and a detailed description of the similar configuration will be omitted.

The transducer holder 100 is connected to the negative pole of the RF board 260 and grounded, and the transducer 20 is connected to the positive pole of the RF board 260 and receives the RF power, as an example.

The transducer holder 100 and the transducers 20 are respectively connected to the board connector 261 of the RF board 260 through the electrode wire.

The electrode wire includes a first electrode wire connecting the transducer holder 100 and the board connector 261, and a second electrode wire 180 connecting the transducer 20 and the board connector 261.

The second electrode wire 180 is a wire that is soldered to the center of the rear surface of the transducer 20 to supply RF power to the transducer 20. The second electrode wire 180 is arranged to pass through the electrode wire hole 120a of the transducer holder 100. The second electrode wire 180 is extended to the rear surface of the ultrasonic radiation frame 10 through the electrode wire hole 120a and connected to the RF board 260.

The RF board 260 is detachably connected to the rear of the ultrasonic radiation frame 10, and is connected to a plurality of first electrode wires each connected to the plurality of transducer holders 100, and a plurality of second electrode wires 180 each connected to the plurality of transducers 20.

The RF board 260 is arranged in the remaining part except the center so as to prevent interference with the probe 11 in the case that the probe 11 is coupled to the rear of the ultrasonic radiation frame 10. In addition, the RF board 260 may be formed in multiple pieces. In this embodiment, four of the RF boards 260 are each in an arc shape and are connected to each other to form a ring shape. When the RF boards 260 are composed of multiple pieces, they may be connected to each other, and of course, they may be arranged at a predetermined distance from each other. In addition, the number and shape of the RF boards 260 may be changed in various ways so long as they may prevent interference with the probe 11. In other words, the RF boards 260 may be changed in various ways so long as they are arranged in the remaining part of the ultrasonic radiation frame 10 except for the central portion, which is the joint portion where the probe 11 is coupled. For example, one or more RF boards 260 may be arranged in shapes such as a square, triangle, and crescent shape in the remaining part of the ultrasonic radiation frame 10 except for the central portion.

In addition, when the RF boards 260 are composed of n pieces, the plurality of transducers 20 may be classified into n groups according to their positions, and the plurality of transducers 20 may be connected to n RF boards 260 for each group. Accordingly, the RF board 260 may be individually replaced and repaired.

The RF board 260 is provided with a plurality of board connectors 261.

The board connector 261 is a connector provided on the RF board 260 to which the first and second electrode wires 180 are detachably connected. The board connectors 261 are formed to correspond to the number of the transducers 20 so that the transducers 20 may be independently connected. However, the present disclosure is not limited thereto, and at least two or more transducers 20 may be connected to one board connector 261, and of course, it is also possible for the number of the board connectors 261 to be greater than the number of the transducers 20. In addition, the board connectors 261 may be integrally provided with the RF board 260, or may be detachably coupled to the RF board 260.

The RF board 260 further includes a monitoring sensor.

The monitoring sensor is a sensor provided with the RF board 260 to independently monitor the operating status of the transducers 20. In this embodiment, the monitoring sensor detects the power supply status of the second electrode wires 180 respectively connected to the transducers 20, thereby detecting normal or abnormal operation of the transducers 20, as an example. For example, the monitoring sensor may be a current sensor or a voltage sensor that detects overcurrent or overvoltage or current cutoff of the electrode wires 180. However, the present disclosure is not limited thereto, and any sensor that may detect an abnormal state of the temperature sensor or the transducers 20 may be applied.

An insulating cover 270 is provided on the outer surface of the RF board 260.

The insulating cover 270 is provided to cover the outer surface of the RF board 260 and serves to insulate. In this embodiment, the insulating cover 270 may be a polyimide film as an example, but it is not limited thereto, and any insulating material may be applied. The insulating cover 270 may be connected to the RF board 260 using a fastening member, and the like, or may be attached to the RF board 260 using a separate adhesive member.

Meanwhile, the RF board 260 is connected to a power supply device for supplying the RF power.

The RF board 260 and the power supply device may be connected to the RF board 260 by a plurality of power cables that are detachably connected. The power cables may be Bayonet Neill-Concelman (BNC) cables equipped with BNC connectors, but are not limited thereto and may be implemented in various ways.

As described above, in this embodiment, the transducer 20 is connected to the RF board 260 and receives the RF power through the RF board 260.

Since the plurality of transducers 20 are respectively connected to the board connectors 261 of the RF board 260, in the case that any one of the plurality of transducers 20 is damaged or needs to be replaced, it is possible to repair or replace only the corresponding transducer 20. That is, in the case that the plurality of electrode wires respectively connected to the transducers 20 are tied together and connected to a separate power supply, there is a problem that not only individually checks the status of the transducers, but also individually repairs or replaces them. On the other hand, in the present disclosure, the RF board 260 is provided between the transducers 20 and the power supply, and the second electrode wires 180 connected to the plurality of transducers 20 are individually connected to the RF board 260 through the board connector, so that individual repair or replacement of the transducers 20 may be made available.

In addition, since the state of the plurality of transducers 20 may be individually monitored using the monitoring sensors provided in the RF board 260, only the transducers 20 that need repair or replacement may be identified more easily and quickly, and a response may be made quickly.

Therefore, since the plurality of transducers 20 may be independently monitored and repaired or maintained, maintenance and management may be made easy.

In addition, since the RF board 260 compatible with the probe 11 may be used, it is easy to treat various types of lesions.

Meanwhile, in the embodiment, the transducer holder 100 is grounded and RF power is applied only to the transducer 20 as an example. However, the present disclosure is not limited thereto, and it is also possible to configure the transducer holder 100 to be connected to the negative pole of the RF board 260 and the transducer 20 to be connected to the positive pole of the RF board 260 so that there is a potential difference between the transducer holder 100 and the transducer 20.

Meanwhile, in the embodiments, the case where the surface of the transducer holder 100 is coated with a conductive material or the entire transducer holder 100 is formed with a conductive material so that the transducer holder 100 and the transducer 20 function as an integral electrode was described as an example. However, the present disclosure is not limited thereto, and the transducer holder 100 may of course be formed of a non-conductive material, that is, an insulating material. In the case that the transducer holder 100 is formed of an insulating material, the first and second electrode wires are respectively connected to the upper and lower ends of the transducer 20, and the first and second electrode wires are connected to the RF board 260.

The present disclosure has been described with reference to the embodiments shown in the drawings, but these are merely exemplary, and those skilled in the art will understand that various modifications and equivalent other embodiments are possible from this. Therefore, the true technical protection scope of the present disclosure should be determined by the technical idea of the appended claims.

According to the present disclosure, a high-intensity focused ultrasound generation device which can minimize noise. may be manufactured.

The high-intensity focused ultrasound generation device according to the present disclosure has the advantage of being able to minimize noise by allowing EMI (Electro Magnetic Interference) noise, which is concentrated only on the transducer, to flow to the ultrasonic radiation frame since the ultrasonic radiation frame, the transducer holder, and the transducer are configured as an integrated electrode, thereby preventing signal loss due to noise and enabling more stable application of RF signals.

In addition, since the ultrasonic radiation frame, the transducer holder, and the transducer are configured as an integrated electrode, the negative pole of the power supply unit can be connected to the negative pole of the ultrasonic radiation frame or a single transducer, thereby greatly simplifying the circuit structure compared to a structure in which each of a plurality of transducers is connected.

In addition, since the transducer holder and the transducer are formed as an integrated electrode, it is possible to minimize sound wave loss during transducer vibration.

In addition, since a plurality of transducers is individually mounted on the ultrasonic radiation frame by the transducer holder, and since the transducer holder and the transducer are electrically connected by contacting each other, there is no need to solder the electrode wire on the front of the transducer, so leakage due to the soldering structure can be prevented, and manufacturing can be made easier.

In addition, since the RF board for applying RF power is provided on the rear of the ultrasonic radiation frame, the plurality of transducers is individually connected to the RF board through the board connector, so that when some of the plurality of transducers requires repair or replacement, it is easy to repair or replace only the corresponding transducer.

In addition, since the monitoring sensor is provided on the RF board, the state of the plurality of transducers can be individually monitored, so there is an advantage in that it is easier to identify a transducer that requires repair or replacement and respond quickly.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A high-intensity focused ultrasound generation device, comprising:
an ultrasonic radiation frame having a coupling hole formed therein;
a transducer holder inserted into the coupling hole and detachably coupled through the ultrasonic radiation frame; and
a transducer mounted on the transducer holder;
wherein the ultrasonic radiation frame, the transducer holder, and the transducer are electrically connected to each other to form an integrated electrode.

2. The device of claim 1, wherein at least one of a negative pole of the ultrasonic radiation frame or the transducer is connected to a negative pole of a connector and grounded; and
wherein the transducer is connected to a positive pole of the connector.

3. The device of claim 1, wherein the ultrasonic radiation frame, the transducer holder, and the transducer comprise a conductive material.

4. The device of claim 3, wherein the conductive material includes at least one of chromium, nickel, cadmium, iron, copper, platinum, gold, silver, lead, or an alloy thereof.

5. The device of claim 1, wherein the ultrasonic radiation frame, the transducer holder, and/or the transducer comprise a non-conductive material and a surface coated with a conductive material.

6. The device of claim 5, wherein the conductive material includes at least one of chromium, nickel, cadmium, iron, copper, platinum, gold, silver, lead, or an alloy thereof.

7. The device of claim 1, wherein the transducer holder includes:
a head portion mounted on the front surface of the ultrasonic radiation frame, wherein the head portion has a mounting groove formed into which the transducer is inserted and mounted; and
a body portion that extends rearward from the head portion, that penetrates the coupling hole, and that is coupled by a fastening member at a rear of the ultrasonic radiation frame.

8. The device of claim 7, wherein the body portion of the transducer holder is formed with a current supply hole through which an electrode wire connected to the transducer passes through and is withdrawn to the rear of the ultrasonic radiation frame; and
wherein a space between the electrode wire and the current supply hole is sealed with waterproof glue.

9. The device of claim 7, wherein the head portion of the transducer holder is formed so that at least a part of a side surface of the mounting groove is opened.

10. The device of claim 7, wherein the head portion of the transducer holder is formed with at least one support protrusion that protrudes from a bottom surface of the mounting groove, that supports a lower surface of the transducer, and that forms a space between the transducer and the bottom surface.

11. The device of claim 7, wherein the head portion of the transducer holder is formed with a catch protrusion that protrudes from a bottom surface of the mounting groove and has a tip bent inward to prevent the transducer inserted into the mounting groove from being detached.

12. The device of claim 7, wherein the body portion of the transducer holder includes:
a shaft portion that extends rearward from the head portion and is press-fitted into the coupling hole; and
a screw portion that extends rearward from the shaft portion, that penetrates the coupling hole, and that is coupled with the fastening member at the rear of the ultrasonic radiation frame.

13. The device of claim 1, wherein the transducer is one of a plurality transducers of the device; and
wherein the device further comprises an RF board, wherein the RF board is configured to electrically connect the plurality of transducers to each other and to supply RF power to the plurality of transducers.

14. The device of claim 13, further comprising:
one or more electrode wires connected to the plurality of transducers; and
one or more board connectors in the RF board corresponding to the one or more electrode wires, and to which the one or more electrode wires are detachably connected.

15. The device of claim 14, wherein the one or more board connectors are detachably connected to the RF board.

16. The device of claim 13, further comprising:
a monitoring sensor provided on the RF board and configured to independently monitor operating states of the plurality of transducers by detecting power supply states of the one or more electrode wires connected to the transducers.

17. The device of claim 13, further comprising:
an insulating cover formed to cover an outer side of the RF board.

18. The device of claim 13, further comprising:
a power supply device for supplying the RF power to the RF board; and
a power cable for connecting the RF board and the power supply device and detachably coupled with the RF board.

19. The device of claim 13, further comprising:
a probe coupled to a center of the ultrasonic radiation frame;
wherein the RF board is provided on a part of the rear of the ultrasonic radiation frame.

20. A high-intensity focused ultrasound generation device, comprising:
an ultrasonic radiation frame having a probe arranged center and a coupling hole;
a transducer holder inserted into the coupling hole and detachably coupled at a rear of the ultrasonic radiation frame; and
a transducer mounted on an open front surface of the transducer holder;
wherein the transducer holder includes:
a head portion mounted on a front surface of the ultrasonic radiation frame, wherein the head portion has a mounting groove formed therein into which the transducer is inserted and mounted; and
a body portion that extends rearward from the head portion, that penetrates the coupling hole, and that is coupled by a fastening member at the rear of the ultrasonic radiation frame;
wherein the head portion has a support protrusion that protrudes from a bottom surface of the mounting groove, that supports a lower surface of the transducer, and that forms a space between the transducer and the bottom surface;
wherein the ultrasonic radiation frame, the transducer holder, and the transducer are electrically connected to each other to form an integrated electrode;
wherein at least one of a negative pole of the ultrasonic radiation frame or the transducer is connected to a negative pole of a connector and grounded,
wherein the transducer is connected to a positive pole of the connector; and
wherein the ultrasonic radiation frame, the transducer holder, and the transducer comprise a conductive material.
